(19) United States
(12) Patent Application Publication
(10) Pub. No.: US 2019/0262190 A1
(43) Pub. Date: Aug. 29, 2019

(54) DISPOSABLE FINGER MOUNTED SWAB

(71) Applicant: Brian A. Fried, Melville, NY (US)

(72) Inventor: Brian A. Fried, Melville, NY (US)

(21) Appl. No.: 15/904,877

(22) Filed: Feb. 26, 2018

(51) Int. Cl. A61F 13/36; A61B 42/20

(52) U.S. Cl. CPC A61F 13/36; A61B 42/20

(57) ABSTRACT

A disposable finger mounted swab has an applicator end configured to contact a body part, natural orifice, open wound or surgical opening of a person or animal and an opening end arranged opposite the applicator end configured for receiving a finger of a user. An outer lining configured for direct contact with the body part, natural orifice, open wound or surgical opening of a person or animal. A water-resistant protective layer attached outer lining. A constricting band attached or integral with outer edges of the opening end of the disposable finger mounted swab and configured to maintain the swab in place on the finger during intended use. The swab may be packaged in a packet, wherein a substance for delivery using the swab is included in or upon the outer lining; the package or packet is hermetically sealed.

### BACKGROUND OF THE INVENTION

The invention broadly relates to cotton swabs and, related more particularly to a disposable, finger mounted swab that is used to safely clean an opening in the body, such as an orifices or a temporary, surgically implemented opening, the swab configured with at least one protective layer and application layer to facilitate mounting upon and use in cooperation with a finger in order to prevent over insertion into an orifice, to enable great sensitivity to applied forces and protection of the finger against contamination by body fluids or medicaments for application by the disposable, finger mounted swab, which is secured to the finger with an elastic band.

Cotton swabs, for example, cotton balls, are known for applying medicaments, conveying nail polish removing fluids, absorbing bodily fluids in medical procedures, etc. But cotton swabs are cumbersome to use, allow the finger or fingers that handle the cotton swab to be exposed to any fluid carried by or otherwise exposed to the cotton swab. Additionally, portions or fibers from the cotton swab, e.g., a cotton ball, at times can become embedded in a surgical opening or orifice, which is uncomfortable.

Cotton swab applicators, such as Q-TIP™ cotton swabs are configured to have a limited and contained amount of cotton fiber at an applicator end of a plastic or wooden stick. While the cotton at the applicator end is configured to maintain its integrity and a swab element, and likely does not leave substantial amounts of cotton fibers in a wound or natural body orifice, their design has inherent disadvantage. For example, because the applicator end is at the end of the applicator stick, which is grasped by the fingers of the user, there is an inherent lack of sensitivity to an amount of force applied. Perhaps more importantly, while instructions for use advise users against inserting the applicator end into an orifice, such as an opening in the human ear, people frequently ignore the warning and insert the applicator end into the ear canal, which can lead to damage.

### SUMMARY OF THE INVENTION

The present invention overcomes the shortcomings of the prior art.

In an embodiment, the invention provides a disposable finger mounted swab has an applicator end configured to contact a body part, natural orifice, open wound or surgical opening of a person or animal and an opening end arranged opposite the applicator end configured for receiving a finger of a user. An outer lining layer with an outer surface and an inner surface and configured for direct contact with the body part, natural orifice, open wound or surgical opening of a person or animal. A water-resistant protective layer is attached the inner surface of the outer lining. A constricting band attached or integral with outer edges of the opening end of the disposable finger mounted swab and configured to maintain the swab in place on the finger during intended use.

The outer lining layer preferably comprises fiber. The fiber is a natural fiber or a man-made fiber. In one embodiment, the fiber is cotton. The outer lining preferably comprises a lining layer and a fiber layer. An inner surface of the water-resistant protective layer further includes a material layer that contacts a user's skin during intended use. The material layer preferably is fiber. For example, cotton. The outer lining layer is preferably hypoallergenic. The water-resistant layer is a barrier. The water-resistant layer prevents communication between the inner side of the barrier and the outer side of the barrier. The constricting band is part of the outer layer. Alternatively, the constricting band is part of the water-resistant layer. Alternatively, the constricting band is formed as part of both the outer lining layer and the water-resistant protective layer. The constricting band operates to seal the opening end against a skin part of an inserted finger, during intended use, e.g., elastic or rubberized.

To use the invention swabs the user slides his/her finger into the opening. Once in place, the user could enter the finger into, for example, an ear canal, just enough to clean without causing damage to ear canal. It is common for people to enter ear canal with cotton on a stick for themselves or their cared ones such as child or elderly and enter the ear or other areas without knowing the limit of how far to go in safely. Other uses for the inventive swabs besides inner and outer ear include the nostrils, cleaning corner of eyes, anal area, wounds imposed by surgery, wounds imposed by accident, etc. without limitation. Of course, the inventive swabs also can be used to apply liquid or cream medicines for people and animals, such as canines and felines. Once used for any of these purposes above, the inventive swabs are disposed of.

The inventive swabs have a cotton liner inside and the outside is a cotton that absorbed and holds the dirt, liquids or creams to hygienically clean the surfaces. The elastic band may embody a thin rubber band like material that is embedded into the bottom of the product to stretch and allow the cotton fingertip to stay in place.

In an embodiment, the inventive swab comes pre-loaded with a substance for delivery in a hermetically-sealed packet. That is, the finger swab includes an outer layer of material, preferably hypoallergenic, such as cotton or organic cotton, infused with a substance for delivery. Under the cotton layer (or other hypoallergenic material layer), is a plastic or shield layer to protect the skin from touching the substance to be delivered. The substance for delivery may be a medicament, for example, a medicated liquid, cream, powder or gel, or a non-medicated liquid, cream, powder or gel, arranged on top of (and/or infused within the hypoallergenic material such as cotton material, to apply).

The user just opens the hermetically-sealed packet, exposes the pre-loaded finger swab, unrolls it down the user's finger and then uses the swab-attached finger to apply the substance. Preferably, the pre-loaded finger swab is disposable, i.e., the uses applies the medicated or non-medicated substance, as the case may be, and then merely disposes of the used swab.

### BRIEF DESCRIPTION OF THE DRAWINGS

The invention will be described in conjunction with the following drawings in which like reference numerals designate like elements and wherein:

FIG. 1 is a perspective view illustrating a first exemplary embodiment of the disposable finger mounted swab of the invention;

FIG. 2 is a second exemplary embodiment of the disposable finger mounted swab of the invention;

FIG. 3A depicts an embodiment of an inventive swab that comes pre-loaded with a substance for delivery in a hermetically-sealed packet; and

FIG. 3B depicts a package or packet that contains the inventive swab embodiment of FIG. 3A.

### DETAILED DESCRIPTION OF THE INVENTION

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

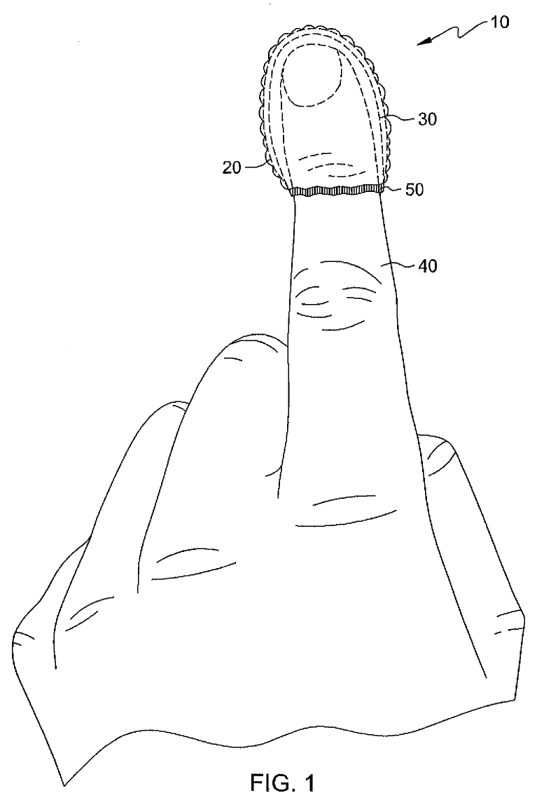

FIG. 1 depicts a first embodiment of the disposable finger mounted swab 10 of the invention. As shown therein the inventive swab 10 includes an outer lining 20 which comes in contact with a body part, natural orifice, open wound or surgical opening of a person or animal. Please note, however, that the exemplary application to a human or animal are meant to be exemplary intended uses, that the inventive swabs may be used for other applications without deviating from the scope and spirit of the invention. Outer lining 20 is preferably cotton, which is a natural fiber, but may be implemented with any fiber, natural or otherwise, known to the skilled artisan to effectively hold to apply a substance, as part of an application function, to absorb fluids or capture particles as part of a cleaning function. Preferably, the outer lining 20 comprises a thin liner base layer to which the fiber is attached or captured with an adhesive, or alternatively attached by first applying a fiber matrix or weave that is then caused to be attached to the thin liner layer.

Under the outer lining 20 is a water-resistant protective layer 30. The water-resistant protective layer 30 is attached to the inner surface of the outer lining 20 and forms a seal between the outer lining 20 and the finer or digit 40 that is inserted into the inventive swab 10 prior to use. The water-resistant protective layer 30 may be formed from any known material, such as latex or plastic that may be applied as a film to the inner surface of the outer lining 20 to form a water-resistant barrier therebetween.

While not shown in the figures, the inventive swabs 10, 60 may include a fiber layer, e.g., cotton, on the inner surface of the water-resistant layer 30, 30′, for the user's comfort.

The inventive swab 10 may be said to have an applicator end and an end opposing the applicator end in which a digit or finger 40 is inserted. To facilitate a seal, or a quasi-seal about a digit or finger during intended use, a constricting band 50 is attached at the edges of the portion of the applicator end with the opening through which the digit or finer 40 is inserted, as shown. Preferably, the constricting band is an elastic element attached to the end edges but is not limited thereto. The constricting band 50 may be rubber and may be attached or integrally formed from the edges.

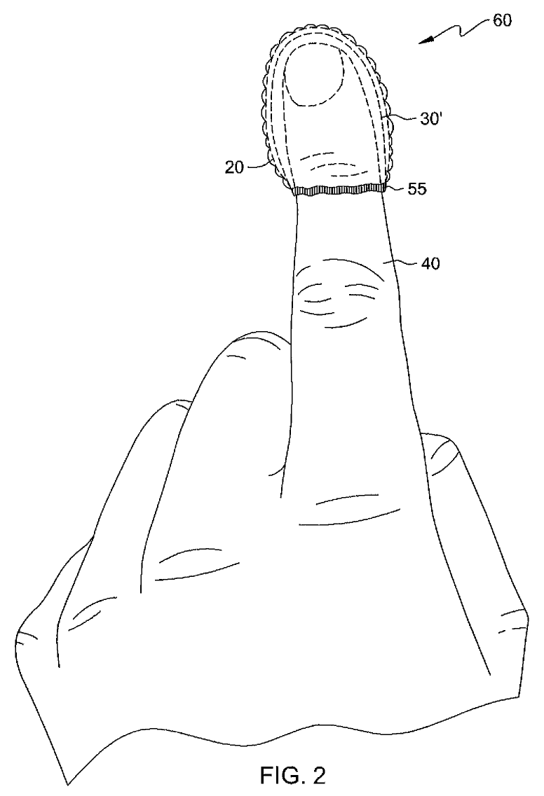

FIG. 2 shows an alternative embodiment of a disposable finger mounted swab 60. The embodiment depicted in FIG. 2 is different than the embodiment in FIG. 1 because the inventive swab 60 includes a water-resistant layer 30′ that has at edges at the opening end of the swab a sealing portion 55 that operates to seal the swab 60 against entry, once a finer or digit is inserted therein. The sealing portion 55 comprises a constricting band, such as constricting band 50 as shown in FIG. 1, and a sealing material proximate the constricting band that operates to adhere the constricting band at the opening end to the users' skin, for a seal that is substantially water-proof, or near water-proof. Once the finger is inserted, the edges must be pressed against the skin about the entire circumference of the end edges of the swab to effect the seal.

The seal prevents any material to be applied or that is being cleaned with or by the inventive swab 10 for entering into a cavity formed and covering a fingertip and contacting the fingertip. This is ideal where the fingertip might have a break in the skin, and skin contact there could cause irritation or infection. Perhaps as importantly, seal the tip of the finger away the applicator end will prevent contamination by the finer to any application site.

Moreover, because of the size of a finer or digit 40, which must be included to use the inventive swab 10, the inventive swab 10 has an inherent safety feature in that it cannot be inserted into an opening or natural orifice further than is allowed by the natural physical dimension of the applying finger or digit. For that matter, and in view of the nerve set presenting in just about every finger or digit used in cooperation with the inventive swab 10 to clean or apply, there is an amount of sensitivity built into any application of force. The tow inherent safety features not only make the inventive swab unique, but also render much safer that an applicator swab of a shaft or even a cotton swab.

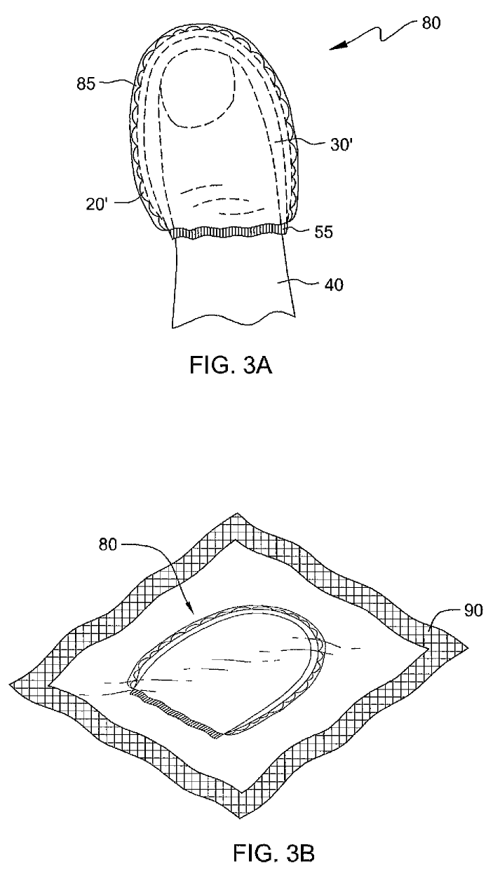

FIGS. 3A and 3B together depict an embodiment of an inventive swab 80 that comes pre-loaded with a substance 85 for delivery in a hermetically-sealed packet 90. That is, the finger swab 80 includes an outer layer of material 20′, preferably hypoallergenic, such as cotton or organic cotton, that is infused with or otherwise carries substance 85. Under the cotton layer (or other hypoallergenic material layer) 20′, is a plastic or shield layer 30′ to protect the skin from touching the substance to be delivered. The substance for delivery 85 may be a medicament, for example, a medicated liquid, cream, powder or gel, or a non-medicated liquid, cream, powder or gel, arranged on top of (and/or infused within the hypoallergenic material such as cotton material, to apply).

The user just opens the hermetically-sealed packet 90, exposes the pre-loaded finger swab 80, unrolls it down the user's finger 40 and then uses the swab-attached finger to apply the substance 85. Preferably, the pre-loaded finger swab 80 is disposable, i.e., the uses applies the medicated or non-medicated substance and then merely disposes of the used swab.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. A disposable finger mounted swab having an applicator end configured to contact a body part, natural orifice, open wound or surgical opening of a person or animal and an opening end arranged opposite the applicator end configured for receiving a finger of a user, comprising:
an outer lining layer with an outer surface and an inner surface and configured for direct contact by the outer surface with the body part, natural orifice, open wound or surgical opening of a person or animal;
a water-resistant protective layer attached the inner surface of the outer lining; and
a constricting band attached or integral with outer edges of the opening end of the disposable finger mounted swab and configured to maintain the swab in place on the finger during intended use.

2. The disposable finger swab as set forth in claim 1, wherein the outer lining layer comprises fiber.

3. The disposable finger swab as set forth in claim 2, wherein the fiber is a natural fiber or a man-made fiber.

4. The disposable finger swab as set forth in claim 2, wherein the fiber is cotton.

5. The disposable finger swab as set forth in claim 1, wherein the outer lining layer comprises an inner lining layer and an outer fiber layer.

6. The disposable finger swab as set forth in claim 5, wherein an inner surface of the water-resistant protective layer further includes material layer that contacts a user's skin during intended use.

7. The disposable finger swab as set forth in claim 6, wherein the material layer of the water-resistant protective layer is fiber.

8. The disposable finger swab as set forth in claim 6, wherein the material layer of the water-resistant protective layer is cotton.

9. The disposable finger swab as set forth in claim 1, wherein the outer lining layer is hypoallergenic.

10. The disposable finger swab as set forth in claim 1, wherein the water-resistant protective layer is a barrier.

11. The disposable finger swab as set forth in claim 10, wherein the water-resistant protective layer prevents communication between the inner side of the barrier and the outer side of the barrier.

12. The disposable finger swab as set forth in claim 1, wherein the constricting band is part of the outer lining layer.

13. The disposable finger swab as set forth in claim 1, wherein the constricting band is part of the water-resistant protective layer.

14. The disposable finger swab as set forth in claim 13, wherein the constricting band operates to seal the opening end against a skin part of an inserted finger, during intended use.

14. The disposable finger swab as set forth in claim 13, wherein the constricting band is elastic.

16. The disposable finger swab as set forth in claim 13, wherein the constricting band is rubberized.

17. A package comprising a disposable finger swab with an outer layer containing or including a substance for application by the disposable finger swab, the disposable finger swab comprising:
an outer material lining layer configured for direct contact with a body part, a natural orifice, an open wound or a surgical opening of a person or animal;
a water-resistant protective layer attached to an inner surface of the outer lining; and
a constricting band attached or integral with outer edges of the outer material lining layer and the water-resistant protective later, at an opening end of the disposable finger swab and configured to maintain the swab in place on the finger during intended use;
wherein the substance is a liquid, cream, powder or gel.

18. The package as set forth in claim 17, wherein the substance is a medicated liquid, cream, powder or gel.

19. The package as set forth in claim 17, wherein the package is formed as a foil or plastic packet.

20. The package as set forth in claim 19, wherein the package is hermetically sealed.
